(19) United States
(12) Patent Application Publication
(10) Pub. No.: US 2021/0186344 A1
(43) Pub. Date: Jun. 24, 2021

(54) CLINICAL ARTIFICIAL INTELLIGENCE (AI) SOFTWARE AND TERMINAL GATEWAY HARDWARE METHOD FOR MONITORING A SUBJECT TO DETECT A POSSIBLE RESPIRATORY DISEASE

(71) Applicants: Pai-chang yeh, Zhubei (TW); Julian Gerald Dcruz, KL (IN)

(72) Inventors: Pai-chang yeh, Zhubei (TW); Julian Gerald Dcruz, KL (IN)

(73) Assignee: Boston Research Corporation, London (GB)

(21) Appl. No.: 17/193,333

(22) Filed: Mar. 5, 2021

Related U.S. Application Data
(60) Provisional application No. 63/130,609, filed on Dec. 25, 2020.

(51) Int. Cl. A61B 5/0205; G06N 3/04; G16H 50/20; G16H 40/67; G16H 30/40; A61B 6/00; A61B 5/00; A61B 5/01; A61B 5/091; A61B 5/05; A61B 5/1468; A61B 5/11; A61B 5/08; A61B 5/1455; A61B 5/021

(52) U.S. Cl. CPC A61B 5/02055; H04L 67/12; G16H 50/20; G16H 40/67; G16H 30/40; A61B 6/44; A61B 5/0077; A61B 5/015; A61B 5/7264; A61B 5/091; A61B 5/05; A61B 5/1468; A61B 5/1126; A61B 5/0816; A61B 5/6804; A61B 5/14551; A61B 5/021; A61B 5/742; A61B 5/0022; G06N 3/04

(57) ABSTRACT

The present invention relates generally to an apparatus and method for detecting diseases and, more, specifically, detecting respiratory diseases such as airborne transmitting diseases at the early stage. The present invention provides a solution in the form of a clinical AI Software and terminal gateway hardware. Terminal gateway with clinical AI software can detect diseases by collecting and analyzing data from multiple sensors and modules. The terminal gateway can offload healthcare professionals from over-work and misjudge due to long working hours. It also provides a better second-opinion for less-experienced professionals. The gateway terminal is used to detect respiratory diseases such as airborne transmitting diseases at the early stage to help offload the healthcare professional in the hospital and to provide an alert when the professionals are not available outside of the hospital. The present invention includes a structure of a gateway or station, multiple sensor modules such as low wattage x-ray, an infrared thermal detector, and the clinical AI software.

### TECHNICAL FIELD

The present invention relates generally to fields of medical diagnostics and prognostics and more specifically, to clinical artificial intelligence (AI) software and terminal gateway hardware method for monitoring a subject to detect a possible respiratory disease.

### BACKGROUND

Background description includes information that may be useful in understanding the present invention. It is not an admission that any of the information provided herein is prior art or relevant to the presently claimed invention, or that any publication specifically or implicitly referenced is prior art.

Severe acute respiratory syndrome (SARS) was first identified in late November 2002 in Guangdong Province, China. In the ensuing months, major outbreaks were reported in other parts of China, Vietnam, Canada, Singapore, Taiwan, and elsewhere in the world. The disease is unusual in its high level of infectivity, as demonstrated among the health care workers and family members that have been in close contact with infected individuals.

The cause of SARS has been identified as a novel coronavirus (CoV) (Drosten et al. (2003) “Identification of a novel coronavirus in patients with severe acute respiratory syndrome,” N. Engl. J. Med. 348:1967-1976, which is incorporated by reference), because clinical specimens from patients infected with SARS revealed the presence of crownshaped CoV particles. This new CoV has thus been referred to as SARS CoV. CoVs are a family of positive-strand RNA-enveloped viruses called Coronaviridae, which are now categorized under the newly established order Nidovirales. This order comprises the families Coronaviridae and Arteriviridae. The name Nidovirales comes from the Latin word nidus, for nest, referring to the 3′-coterminal “nested” set of subgenomic mRNAs produced during viral infection (Cavanagh (2003) “Nidovirales: a new order comprising Coronaviridae and Arteriviridae,” Arch. Virol. 14:629-633, which is incorporated by reference).

Coronavirus disease 2019 (COVID-19) is a contagious disease caused by severe acute respiratory syndrome coronavirus 2 (SARS-CoV-2). The first case was identified in Wuhan, China, in December 2019. It has since spread worldwide, leading to an ongoing pandemic. The COVID-19 pandemic, also known as the coronavirus pandemic, is an ongoing pandemic of coronavirus disease 2019 (CO VID-19) caused by severe acute respiratory syndrome coronavirus 2 (SARS-CoV-2).

With the outbreak of the COVID-19 virus, the health system came to a realization of the shortage of skilled personnel and equipment in hospitals for efficient diagnosis of diseases. Also, the need for contactless examination for efficient diagnosis of diseases arises with the outbreak of COVID-19.

There remains a need to develop monitoring and detection systems that can gather and analyze data for detecting respiratory diseases such as airborne transmitting diseases at the early stage.

As used in the description herein and throughout the claims that follow, the meaning of “a,” “an,” and “the” includes plural reference unless the context clearly_{y} dictates otherwise, Also, as used in the description herein, the meaning of “in” includes “in” and “on” unless the context clearly dictates otherwise.

### SUMMARY

The present invention overcomes the above-described and other problems and disadvantages in the prior art.

The present invention relates generally to an apparatus and method for detecting diseases and, more specifically, detecting respiratory diseases such as airborne transmitting diseases at the early stage.

The present invention provides a solution in the form of a clinical AI Software and terminal gateway hardware. Terminal gateway with clinical AI software can detect diseases by collecting and analyzing data from multiple sensors and modules.

The terminal gateway can offload healthcare professionals from over-work and misjudge due to long working hours. It also provides a better second-opinion for less-experienced professionals.

The present invention can be used outside of hospitals at places such as but not limited to transportation hubs like airports or train stations. The terminal gateway can be a quick alert to remind non-healthcare professionals about potential infectious diseases like COVID-19. Then they can protect themselves and handle the suspicious patients carefully.

The gateway terminal is used to detect respiratory diseases such as airborne transmitting diseases at the early stage to help offload the healthcare professional in the hospital and to provide an alert when the professionals are not available outside of the hospital.

The present invention includes a structure of a gateway or station, multiple sensor modules such as low wattage x-ray, an infrared thermal detector, and the clinical AI software.

The present invention provides:
i. Quick detection of airborne transmitting diseases,
ii. High accuracy of the detection result,
iii. Protection to patient privacy,
iv. Mobility of the portable facility,
v. Lower cost than medical imaging equipment, and
vi. Updatable AI software module.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 is a simplified perspective view of a preferred embodiment incorporating the system to monitor a subject to detect possible respiratory diseases.

FIG. 2 illustrates an exemplary forms of implementation that the present invention can be implemented.

FIG. 3 presents a diagrammatic view of one preferred arrangement illustrating the components of the system of the present invention.

FIG. 4A illustrates components of the terminal gateway, according to another embodiment of the present invention.

FIG. 4B illustrates data capture flow, according to another embodiment of the present invention.

FIG. 4C illustrates a data sync flow, according to another embodiment of the present invention.

FIG. 5 illustrates a system or apparatus for monitoring a subject to detect a possible respiratory disease in the monitored subject, according to another embodiment of the present invention.

FIG. 6 illustrates a method for monitoring a subject to detect a possible respiratory disease in the monitored subject, according to another embodiment of the present invention.

### DETAILED DESCRIPTION

Embodiments of the present disclosure include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware or by human operators.

The following detailed description is made with reference to the technology disclosed. Preferred implementations are described to illustrate the technology disclosed, not to limit its scope, which is defined by the claims. Those of ordinary skill in the art will recognize a variety of equivalent variations on the description.

Examples of systems, apparatus, computer-readable storage media, and methods according to the disclosed implementations are described in this section. These examples are being provided solely to add context and aid in the understanding of the disclosed implementations. It will thus be apparent to one skilled in the art that the disclosed implementations may be practiced without some or all of the specific details provided. In other instances, certain process or method operations, also referred to herein as “blocks,” have not been described in detail in order to avoid unnecessarily obscuring the disclosed implementations. Other implementations and applications also are possible, and as such, the following examples should not be taken as definitive or limiting either in scope or setting.

In the following detailed description, references are made to the accompanying drawings, which form a part of the description and in which are shown, by way of illustration, specific implementations. Although these disclosed implementations are described in sufficient detail to enable one skilled in the art to practice the implementations, it is to be understood that these examples are not limiting, such that other implementations may be used and changes may be made to the disclosed implementations without departing from their spirit and scope. For example, the blocks of the methods shown and described herein are not necessarily performed in the order indicated in sonic other implementations. Additionally, in sonic other implementations, the disclosed methods may include more or fewer blocks than are described. As another example, some blocks described herein as separate blocks may be combined in some other implementations. Conversely, what may be described herein as a single block may be implemented in multiple blocks in some other implementations. Additionally, the conjunction “or” is intended herein in the inclusive sense where appropriate unless otherwise indicated; that is, the phrase “A, B or C” is intended to include the possibilities of “A,” “B,” “C,” “A and B,” “B and C,” “A and C” and “A, B and C.”

Some implementations described and referenced herein are directed to systems, apparatus, computer-implemented methods and computer-readable storage media for detecting flooding of message queues.

Thus, for example, it will be appreciated by those of ordinary skill in the art that the diagrams, schematics, illustrations, and the like represent conceptual views or processes illustrating systems and methods embodying this disclosure. The functions of the various elements shown in the figures may be provided through the use of dedicated hardware as well as hardware capable of executing associated software. Similarly, any electronic code generator shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular named.

Various terms as used herein are shown below. To the extent a term used in a claim is not defined below, it should be given the broadest definition persons m the pertinent art have given that term as reflected in printed publications and issued patents at the time of filing.

The present invention relates generally to an apparatus and method for detecting diseases and, more specifically, detecting respiratory diseases such as airborne transmitting diseases at the early stage.

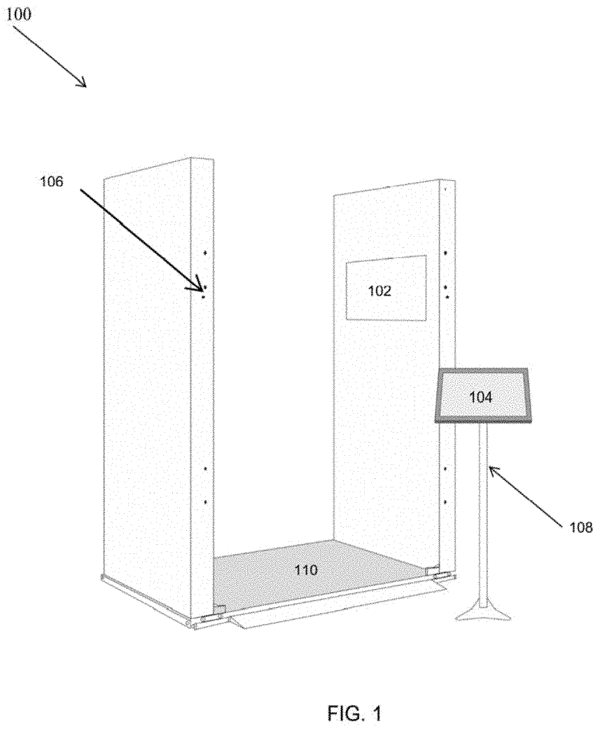

Referring to the drawings in detail, FIG. 1 illustrates one proposed embodiment deploying the present invention 100. Multiple sensors (106) (herein after interchangeable referred to as “collectors”) would be located and mounted on a known walk-through gateway, such as shown in FIG. 1. Each collector may include an air vacuum or be attached to an air vacuum which would gather and pull in air surrounding the collector. Accordingly, as an individual (not shown in outline form) passed through the walk-through gateway, air would be sampled in the immediate vicinity of the individual passing therethrough. By way of example and not by way of limitation, the collectors might he located approximately 25 to 50 cm away from the individual. The particular location would vary depending on the mounting location and depending on the sensitivity of the collector. The entire arrangement will be contactless for the individual.

Each collector would be connected by a tube or passageway to a sensor or a plurality of sensors located nearby. Accordingly, an airborne specimen is obtained.

Once the collectors have gathered an airborne specimen or sample, the particulate matter in the specimen will be analyzed by the sensor or plurality of sensors. As the scanned for information is obtained, it will be transmitted to a central location.

A transmitting system having central processing unit will communicate information to a central information gathering location. At the central information gathering location, a monitoring system CPU validates the information, and provides for historical or precipitating data analysis. The process to collect data may follow a process having a number of steps. In an initialization step, alert monitoring software will be retrieved from memory of the monitoring system CPU. Thereafter subject/persons to be monitored will be determined. The monitoring system CPU will thereafter be in a ready state awaiting communication from a transmitting system CPU.

The system will be fitted with one or more screens inside 102 or outside 104 of the, tunnel or gateway may or may not he on the stand 108, where the internal screen will displayed the results to the user and the outside display will display the results to the supervisor, lab attendant or other person who is supervising the person entering the premises. In an example, when a user steps on the platform 110, the sensors 106 collects the user breadth and analyzes the collected particles and display the results to the user on the internal screen 102 and to the other person who is monitoring the users on the second screen 104.

A benefit of the present invention is that it could be employed with existing metal detectors in place which would be in close proximity to those passing into and through airports and government buildings. Accordingly, the structure for deploying such a system is already in place.

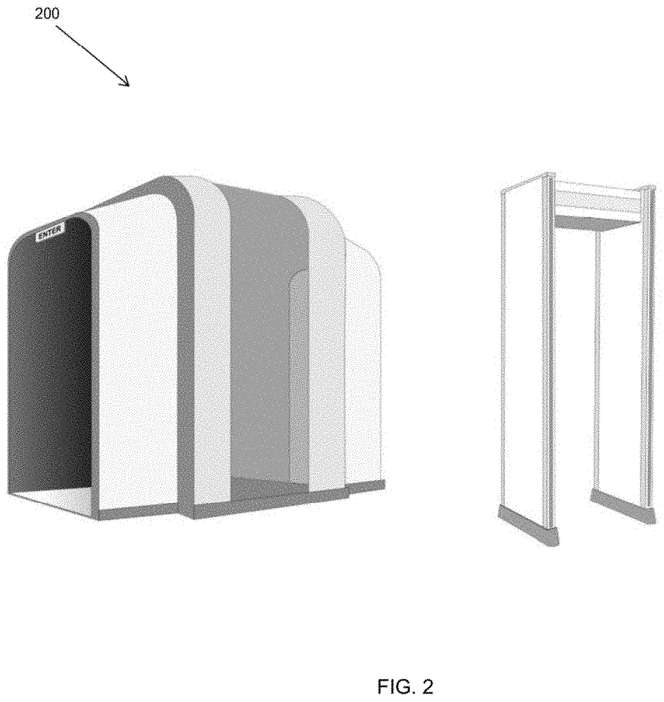

FIG. 2 illustrates an exemplary form of implementations 200, that the present invention can be implemented. In this form of implementation, the system consists of a tunnel or gateway like structure to detect the respiratory diseases. The system or arrangement is completely contactless.

The form of implementation of such arrangement varies in shape and size depending upon the application and place of use. Also, the tunnel may be made collapsible or extendable.

The size of the tunnel or gateway may be increased or reduced depending upon the application of use, where the size can be increased to maintain social distancing while entering the premises and avoid capturing the respiration of another individual.

Also, in other implementation, the tunnel or gateway may be made for entering only one individual and automatic sanitization of the tunnel/gateway with quick pressured stream of sanitization.

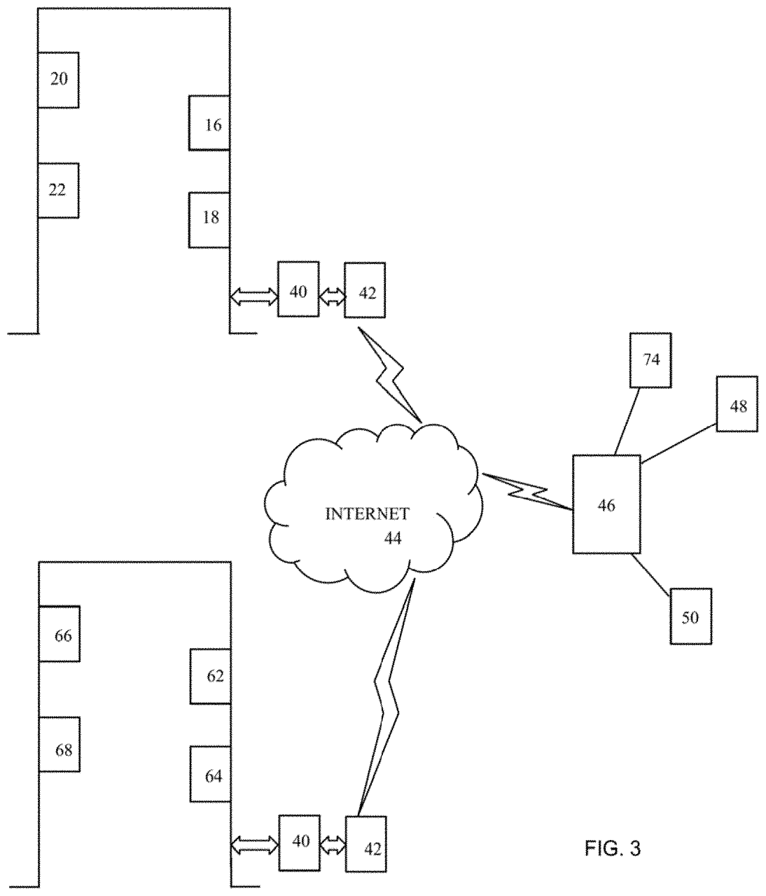

FIG. 3 illustrates a diagrammatic view of one arrangement showing the components of a system 10 to monitor, detect and analyze as set forth u the present invention. A structure such as a known metal gateway 12 may have incorporated thereon a number of sensor collectors 16, 18, 20, and 22 which would be mounted thereon. Once the collectors have gathered an airborne specimen by means of a vacuum, the specimen will be analyzed by a sensor or sensors 40. The sensors 40 are replaceable so that a failure of any sensor could be addressed by simple replacement of the sensor. The sensors may be so-called “plug and play”, allowing simple and robust connection with other devices by common protocols and procedures following universal standards, so that devices may be connected without additional programming. The sensor 40 will generate electronic signals or alerts which will be delivered to a transmitting or monitored central processing unit 42.

The transmitting or monitored central processing unit 42 will be connected to a network, such as the Internet 44 or standard telecommunication networks, and thereafter the data will be delivered to a central site CPU 46.

Levels of encryption are applied to all data transfer. User authentication must occur before the connection between the transmitting central processing unit and central site CPU 46 will be established. This requires the user to enter a unique ID and password, which must be approved by the target machine. The system's embedded security features inhibits the possibilities for intrusion and the willful interjection of false positives. The central site CPU 46 will, in turn, be in contact with a government agency 48 or a responder such as the Center for Disease Control.

Once the sensors have gathered an airborne specimen by means of a vacuum, the specimen will be analyzed. The analysis will result in sending alert data to a central processing unit 46. The central processing unit 46 is a transmitting or monitored central processing unit which is connected through the Internet 44 to a central site CPU 46. In this way, multiple sensors can gather data from multiple locations such as large office buildings and airports.

Each of the transmitting or monitored system CPUs 42 operate under the control of an operating system, such as a Linux operating system, which facilitates requests made by central site CPU software. The operating system will also have application programs in a client-server format. Various types of alert monitoring software are known to those skilled in the art and may include any number of third party offerings. Examples of such third party alert monitoring programs include, but are not, limited to, Omegan, Tivoli or TNG.

It will be understood herein that while the description of an alarm is made, no physical, visual or audible alarm may be made. Accordingly, the sensors operate transparently to those passing by.

Data will be correlated by the central site CPU 46 for analysis. The data will also be subject to a number of tests. For example, the data may be tested for redundancy. The data may also be checked for reasonableness.

Communication between the monitored system CPUs 42 and the monitoring system central site CPU 46 is also known to those skilled in the art. Communication can be facilitated by a network, such as the World Wide Web, or any other network configuration supporting inter-computer communication. A secure connection can be established in various ways.

For example, in one arrangement contemplated herein, the transmitting or monitored CPU 42 will retrieve a dynamic address by contacting a secure name server utilizing a unique combination ID/password which itself is encrypted. The transmitting or monitored CPU is then able to present an authorized user ID/password to a mail server and securely logon.

The central site or monitoring CPU system 46 will also obtain a dynamic address by contacting the secure name server utilizing a unique combination ID/password which is itself encrypted. The monitoring CPU is then able to present an authorized user ID/password to the mail server and log on.

In one deployment of the present invention, the system would be non-intrusive and non-invasive. For example, an individual passing a metal gateway at an airport would not be specifically identified. At the same time, data gathered can be correlated and analyzed. Again by way of example, the number of airline passengers traveling from Hong Kong to San Francisco carrying influenza could be identified.

In an essential embodiment, the present invention relates generally to an apparatus and method for detecting diseases and, more specifically, detecting respiratory diseases such as airborne transmitting diseases at the early stage.

The present invention provides a solution in the form of a clinical AI Software and terminal gateway hardware. Terminal gateway with clinical AI software can detect diseases by collecting and analyzing data from multiple sensors and modules.

The gateway terminal is to detect respiratory diseases such as airborne transmitting diseases at the early stage to help offload the healthcare professional in the hospital and to provide an alert when the professionals are not available outside of the hospital.

### A. Clinical AI Software

The Clinical AI Software is developed in C++ and Python utilizing the system hardware drivers to the full core. For the initial experiments, open dataset of X-ray images of the lungs may be used for the AI model to learn the diseases. Later on, more image data from hospitals may be acquired and the AI model may be used to differentiate the subcategories of the target diseases.

Because the region of interest in the image is pretty small and the variance in the image between patients having the disease and healthy people is not evident. A custom AI model with 5 convolution blocks and the parameters are tuned to take in the dataset and learn the features in the images.

In an exemplary embodiment, Convolutional Neural Networks (CNN) are analogous to traditional ANNs in that they are composed of neurons that self-optimize through learning. Each neuron will still receive inputs and perform operations (such as a scalar product followed by a nonlinear function)—the basis of countless ANNs. From the input raw image vectors to the final output of the class score, the entire network will still express a single perceptive score function (the weight). The last layer will contain loss functions associated with the classes, and all of the regular tips and tricks developed for traditional ANNs still apply. For image based deep learning, convolution neural networks (CNN) have been used in solving problems ranging from fault detection to typical digit classification problems.

An augmentation method is used to re-scale the input dataset and additional rotational shift so that Feature Learning is made efficient at the time of training. The input is received as 150×150 and features are extracted, a 3×3 sliding window is used in each convolution layer.

The AI model is utilized in the Clinical AI Software for giving an accurate diagnosis from a patient's imaging data. Along with the features that are learned by the AI model and thereby utilize informational data that is displayed as the output in the Clinical AI Software. The Clinical AI Software also provides a Feature Analyser that highlights the regions in the image with visual colors to help the health professional to confirm the diagnosis provided by the Software.

The in-house data learning is also part of Clinical AI software. If the healthcare professional has found the diagnosis misjudged, then he can mark the data for review. The batch of review images is then automatically used for incremental learning for the AI model. The model learns the features from the review images and improves the accuracy.

The algorithm for the In-house Data Learning' provides the capability to continue the training process on the trained model utilizing the saved callback parameters. As a result, the data are kept in the hospital to conform to the data privacy. The Clinical AI software connects to the Docsun Clinical AI server to get the updates of the new version and uploads reports and model data.

### B. Terminal Gateway Hardware

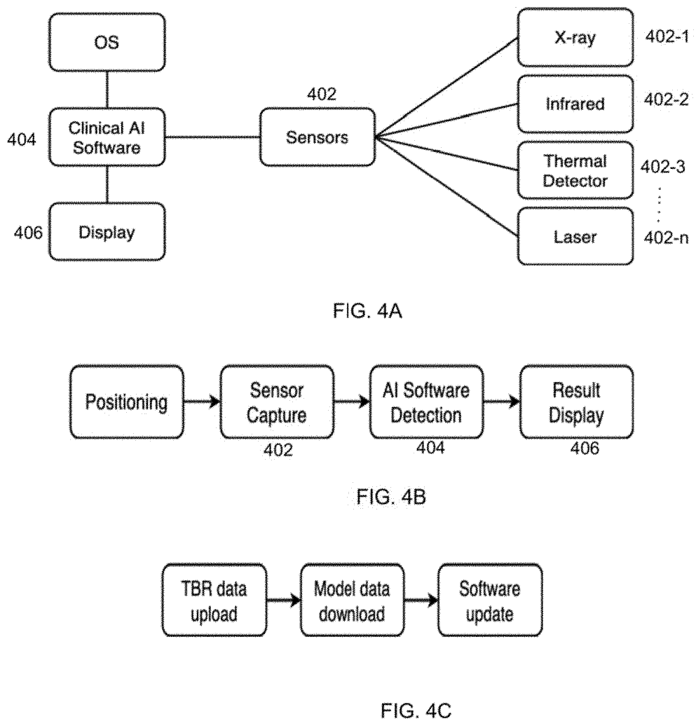

The Terminal Gateway consists of the following parts illustrated in FIG. 4A
i. The structure of a gateway in but not limited to the forms of FIGS. 1-2.
ii. multiple sensor modules include but not limited to a low wattage X-ray, an infrared camera, a thermal detector, and a low-power laser;
iii. the operating system with the Clinical AI software; and
iv. A display device.

FIG. 4A illustrates components of the terminal gateway, according to another embodiment of the present invention.

The structure of the gateway provides support for the sensor modules 402-1, 402-2, 402-3 and . . . , 402-n (hereinafter collectively referred to as sensors 402) and the display 406. There is a wheel system with the braking function to keep the gateway easily movable and fixed. There are two sides to the structure. One is the transmitting side and the other is the receiving side. The sensor modules are embedded at the transmitting side and the receiving side is to get imaging such as X-ray.

The multiple sensor modules will capture the imaging data from the low wattage X-ray, the infrared thermal detector, and the low-power laser. The infrared camera and the laser can also provide feedback to the passenger and help keep the correct position for data capture. The chest images captured will be sent to the Clinical AI software 404 for analysis and detection.

The operating system can be either Linux- or Windows-based. The Clinical AI Software and its database are running in the operating system. Clinical AI Software is described above.

The display device is o show the instruction to the passenger and the detection result to the administrator.

### C. The Workflow of the System

FIG. 4B illustrates data capture flow, according to another embodiment of the present invention. The data capture flow includes the following steps:
i. Positioning: system shows instruction and visual/audio feedback to help the passenger keep correct gestures and position.
ii. Capturing: the multiple sensor modules 402 capture the imaging data.
iii. Detecting: Clinical AI Software 404 runs the AI algorithm to analyze the imaging data and determine the detection result.
iv. Displaying: the display device 406 shows the result of the detection. If the result is positive of any of the target diseases, the device alerts the administrator by visual and audio.

FIG. 4C illustrates a data sync flow, according to another embodiment of the present invention. The data sync flow includes the following steps to connect to the Docsun AI Server regularly and keeps the model data in sync:
i. Uploading the data marked as to Clinical AT Software will connect to the Docsun AI Server and upload the marked images for further investigation on the server-side. (This step is optional according to the administration setting and hospital policy)
ii. Synchronizing the model data: Clinical AI Software uploads the local model data if the accuracy is higher than the initial status of the synchronization cycle. It then downloads the aggregated model data from the server-side to update local model data.
iii. Checking for software update: Clinical AI Software will check if there is a newer version of the software or components that require to be updated. It downloads the new version packages and upgrades itself.

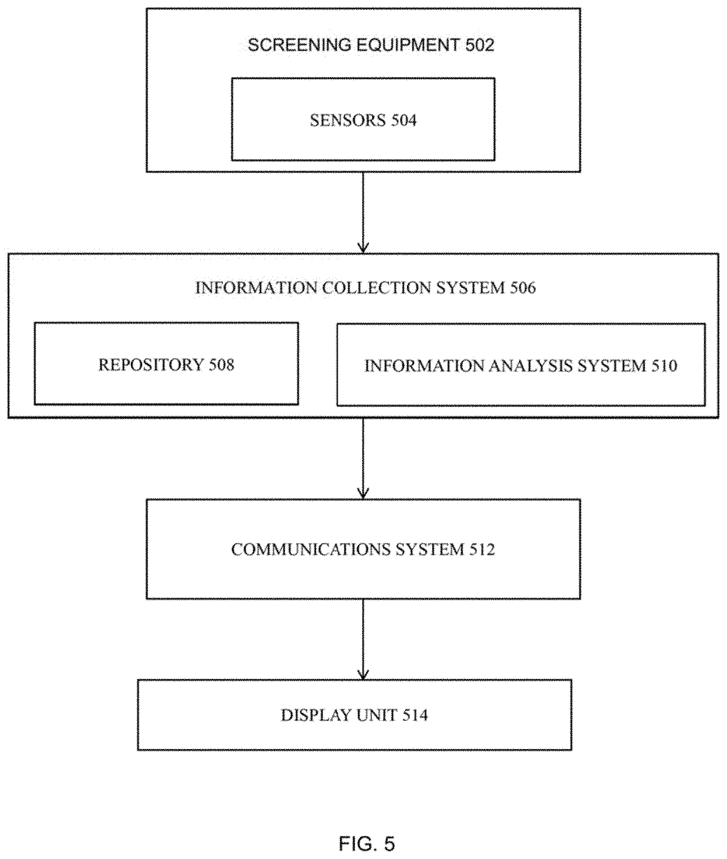

FIG. 5 illustrates a system or apparatus for monitoring a subject to detect a possible respiratory disease in the monitored subject, according to another embodiment of the present invention.

In an exemplary embodiment, a system for monitoring a subject to detect a possible respiratory disease in the monitored subject is provided. The system includes:
a plurality of sensors 504 located in or mounted on a screening equipment 502 wherein each of the plurality of sensors gathers data on one or more physiological parameters associated with the subject to be monitored;
an information collection system 506 comprising at least one repository 608 having information and data obtained from at least one information source, the information and data being identified from the at least one information source by searching the at least one information source for the one or more gathered physiological parameters,
the information collection system 506 further comprising an information analysis system 510 to analyze the searched information source for the one or more gathered physiological parameters for detecting the possible respiratory disease in the monitored subject;
a communications system 512 for transmitting at least a portion of a report generated based on analysis on the display 514, the portion at least indicate a presence or an absence of the possible respiratory disease in the monitored subject

In an exemplary embodiment, the one or more sensors include any one or more of: an X-ray sensor, an infrared thermal detector, a laser sensor, and a camera sensor.

In an exemplary embodiment, the gathered data comprises an imaging data associated with the subject to be monitored, the gathered imaging data associated with the subject to be monitored is compared with the information and data being identified from the at least one information source by searching the at least one information source for detecting the possible respiratory disease in the monitored subject, and the portion of a report generated based on analysis comprises a highlighted gathered imaging data indicating one or more regions in the image with one or more visual colors.

In an exemplary embodiment, the gathered imaging data is feed to an image-based neural network for detecting the possible respiratory disease in the monitored subject, the neural network comprises machine learning based quality prediction models selected from the group including of convolution neural networks (CNN), support vector machine (SVM), artificial neural networks (ANNs), neurofuzzy classifier (NFC), and neuro-wavelet technique (NWT).

In an exemplary embodiment, the communications system include an artificial intelligence model to generate the at least the portion of the report

In an exemplary embodiment, the information analysis system includes an artificial intelligence model to analyze the searched information source for the one or more gathered physiological parameters.

In an exemplary embodiment, the one or more sensors include any one or more of: a respiration sensor, a continuous spirometer, a radio frequency (RF) non-contact sensor, a biomotion sensor, a biological, and a chemical sensor.

In an exemplary embodiment, the one or more sensors include any one or more of: a wearable sensor, pressure sensors, acoustic sensors, humidity sensors, oximetry sensors, acceleration sensors, resistive sensors, and a breathing sensor, such as an accelerometer dipped to the belt or bra, a chest-band (e.g., the spire.io device), a nasal cannula, or extraction of the waveform from a PPG (photoplethysmography) signal.

In an exemplary embodiment, the screening equipment comprises any one or more of a metal detector, a millimeter wave machine, a backscatter x-ray, a cabinet x-ray machine, and a transmission (Penetrating) X-ray security scanner

In an exemplary embodiment, the one or more physiological parameters are selected from the group consisting of: breathing rate, periodic breathing, an amplitude of breathing, an absence of respiration, dominant respiratory frequency, respiratory power, heart rate, blood pressure, a variability of heart rate, a blood oxygen level, and a motion profile.

In an exemplary embodiment, the system includes a display unit 514 for displaying the report generated.

In another embodiment, screening equipment for monitoring a subject to detect a possible respiratory disease in the monitored subject is provided. The screening equipment includes:
a plurality of sensors located in or mounted on the screening equipment, wherein each of the plurality of sensors gathers imaging data associated with the subject to be monitored;
an information collection system comprising at least one repository having information and data obtained from at least one information source, the information and data being identified from the at least one information source by searching the at least one information source for the gathered imaging data,
the information collection system further comprising an information analysis system to analyze the searched information source for the gathered imaging data for detecting the possible respiratory disease in the monitored subject; and
a communications system for transmitting at least a portion of a report generated based on analysis, the portion at least indicate a presence or an absence of the possible respiratory disease in the monitored subject.

In an exemplary embodiment, the one or more sensors include any one or more of: an X-ray sensor, an infrared thermal detector, a laser sensor, and a camera sensor.

In an exemplary embodiment, the portion of a report generated based on analysis comprises a highlighted gathered imaging data indicating one or more regions in the image with one or more visual colors.

In an exemplary embodiment, the gathered imaging data is feed to an image-based neural network for detecting the possible respiratory disease in the monitored subject, the neural network comprises machine learning based quality prediction models selected from the group including of convolution neural networks (CNN), support vector machine (SVM), artificial neural networks (ANNs), neurofuzzy classifier (NFC), and neuro-wavelet technique (NWT).

In an exemplary embodiment, the communications system comprises an artificial intelligence model to generate the at least the portion of the report.

In an exemplary embodiment, the information analysis system comprises an artificial intelligence model to analyze the searched information source for the one or more gathered physiological parameters.

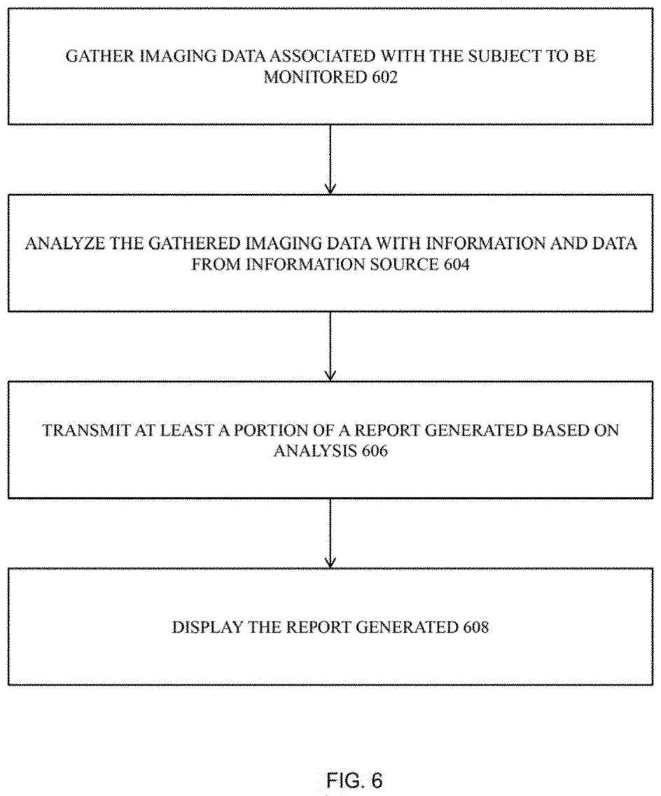

FIG. 6 illustrates a method for monitoring a subject to detect a possible respiratory disease in the monitored subject, according to another embodiment of the present invention.

At step 602, a plurality of sensors located in or mounted on the screening equipment gathers imaging data associated with the subject to be monitored.

At step 604, an information analysis system analyzes a searched information source for the gathered imaging data for detecting the possible respiratory disease in the monitored subject. An information collection system includes at least one repository having information and data obtained from at least one information source, the information and data being identified from the at least one information source by searching the at least one information source for the gathered imaging data.

At step 606, a communications system for transmitting at least a portion of a report generated based on analysis, the portion at least indicate a presence or an absence of the possible respiratory disease in the monitored subject.

At step 608, a display unit for displaying the report generated.

Although the proposed system has been elaborated as above to include all the main modules, it is completely possible that actual implementations may include only a part of the proposed modules or a combination of those or a division of those into sub-modules in various combinations across multiple devices that can be operatively coupled with each other, including in the cloud. Further the modules can be configured in any sequence to achieve objectives elaborated. Also, it can be appreciated that proposed system can be configured in a computing device or across a plurality of computing devices operatively connected with each other, wherein the computing devices can be any of a computer, a laptop, a smartphone, an Internet enabled mobile device and the like. All such modifications and embodiments are completely within the scope of the present disclosure.

As used herein, and unless the context dictates otherwise, the term “coupled to” is intended to include both direct coupling (in which two elements that are coupled to each other or in contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices are able to exchange data with each other over the network, possibly via one or more intermediary device.

Moreover, in interpreting both the specification and the claims, all terms should be interpreted in the broadest possible manner consistent with the context. In particular, the terms “comprises” and “includes” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the protection scope of the appended claims.

What is claimed is:

1. A system for monitoring a subject to detect a possible respiratory disease in the monitored subject, the system comprising:
a plurality of sensors located in or mounted on a screening equipment, wherein each of the plurality of sensors gathers data on one or more physiological parameters associated with the subject to be monitored;
an information collection system comprising at least one repository having information and data obtained from at least one information source, the information and data being identified from the at least one information source by searching the at least one information source for the one or more gathered physiological parameters,
wherein the information collection system further comprising an information analysis system to analyze the searched information source for the one or more gathered physiological parameters for detecting the possible respiratory disease in the monitored subject; and
a communications system for transmitting at least a portion of a report generated based on analysis, the portion at least indicate a presence or an absence of the possible respiratory disease in the monitored subject.

2. The system of claim 1, wherein the one or more sensors comprises any one or more of: an X-ray sensor, an infrared thermal detector, a laser sensor, and a camera sensor.

3. The system of claim 1, wherein:
the gathered data comprises an imaging data associated with the subject to be monitored,
the gathered imaging data associated with the subject to be monitored is compared with the information and data being identified from the at least one information source by searching the at least one information source for detecting the possible respiratory disease in the monitored subject, and
the portion of a report generated based on analysis comprises a highlighted gathered imaging data indicating one or more regions m the image with one or more visual colors.

4. The system of claim 3, wherein the gathered imaging data is feed to an image-based neural network for detecting the possible respiratory disease in the monitored subject, the neural network comprises machine learning based quality prediction models selected from the group including of convolution neural networks (CNN), support vector machine (SVM), artificial neural networks (ANNs), neurofuzzy classifier (NFC), and neuro-wavelet technique (NWT).

5. The system of claim 1, wherein the communications system comprises an artificial intelligence model to generate the at least the portion of the report.

6. The system of claim 1, wherein the information analysis system comprises an artificial intelligence model to analyze the searched information source for the one or more gathered physiological parameters.

7. The system of claim 1, wherein the one or more sensors comprises any one or more of: a respiration sensor, a continuous spirometer, a radio frequency (RF) non-contact sensor, a biomotion sensor, a biological, and a chemical sensor.

8. The system of claim 1, wherein the one or more sensors comprises any one or more of: a wearable sensor, pressure sensors, acoustic sensors, humidity sensors, oximetry sensors, acceleration sensors, resistive sensors, and a breathing sensor, such as an accelerometer clipped to the belt or bra, a chest-band (e.g., the spire.io device), a nasal cannula, or extraction of the waveform from a PPG (photoplethysmography) signal.

9. The system of claim 1, wherein the screening equipment comprises any one or more of a metal detector, a millimeter wave machine, a backscatter x-ray, a cabinet x-ray machine, and a transmission (Penetrating) X-ray security scanner.

10. The system of claim 1, wherein the one or more physiological parameters are selected from the group consisting of: breathing rate, periodic breathing, an amplitude of breathing, an absence of respiration, dominant respiratory frequency, respiratory power, heart rate, blood pressure, a variability of heart rate, a blood oxygen level, and a motion profile.

11. The system of claim 1, further comprising: a display unit for displaying the report generated.

12. A screening equipment for monitoring a subject to detect a possible respiratory disease in the monitored subject, the screening equipment comprising:
a plurality of sensors located in or mounted on the screening equipment, wherein each of the plurality of sensors gathers imaging data associated with the subject to be monitored;
an information collection system comprising at least one repository having information and data obtained from at least one information source, the information and data being identified from the at least one information source by searching the at least one information source for the gathered imaging data,
wherein the information collection system further comprising an information analysis system to analyze the searched information source for the gathered imaging data for detecting the possible respiratory disease in the monitored subject; and
a communications system for transmitting at least a portion of a report generated based on analysis, the portion at least indicate a presence or an absence of the possible respiratory disease in the monitored subject.

13. The screening equipment of claim 12, wherein the one or more sensors comprises any one or more of: an X-ray sensor, an infrared thermal detector, a laser sensor, and a camera sensor.

14. The screening equipment of claim 12, wherein the portion of a report generated based on analysis comprises a highlighted gathered imaging data indicating one or more regions in the image with one or more visual colors.

15. The screening equipment of claim 12, wherein the gathered imaging data is feed to an image-based neural network for detecting the possible respiratory disease in the monitored subject, the neural network comprises machine learning based quality prediction models selected from the group including of convolution neural networks (CNN), support vector machine (SVM), artificial neural networks (ANNs), neurofuzzy classifier (NFC), and neuro-wavelet technique (NWT).

16. The screening equipment of claim 12, wherein the communications system comprises an artificial intelligence model to generate the at least the portion of the report.

17. The screening equipment of claim 12, wherein the information analysis system comprises an artificial intelligence model to analyze the searched information source for the one or more gathered physiological parameters.

18. The screening equipment of claim 12, further comprising: a display unit for displaying the report generated.

19. A method for monitoring a subject to detect a possible respiratory disease in the monitored subject, the method comprising:
gathering, by a plurality of sensors located in or mounted on a screening equipment, imaging data associated with the subject to be monitored;
analyzing, by an information analysis system, the gathered imaging data with an information and data from at least one information source present in at least one repository to detect the possible respiratory disease in the monitored subject, wherein the information and data being identified from the at least one information source by searching the at least one information source for the gathered imaging data;
transmitting, by the communications system, at least a portion of a report generated based on analysis, the portion at least indicate a presence or an absence of the possible respiratory disease in the monitored subject.

20. The method of claim 19, further comprising: displaying, at a display unit, the report generated.
